(19) United States
(12) Patent Application Publication
(10) Pub. No.: US 2008/0293157 A1
(43) Pub. Date: Nov. 27, 2008

(54) Apparatus and method of performing high-throughput cell-culture studies on biomaterials

(76) Inventors: Gerald Frederickson, Westford, MA (US); Adrian McNamara, Galway (IE); Rachit Ohri, Framingham, MA (US); Todd R. Robida, Southbridge, MA (US); Anne M. Whalen, Medway, MA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/753,063

(22) Filed: May 24, 2007

(51) Int. Cl. G01N 1/28; C12M 1/00; C12M 1/34

(52) U.S. Cl. 436/174; 435/283.1; 435/288.4

(57) ABSTRACT

In one embodiment, a kit includes a base and a specimen removably couplable to the base. A top plate defines a plurality of apertures and is removably couplable to the base such that at least two of the apertures are associated with a specimen and each aperture defines a well with that specimen. Each well is configured to receive a sample material therein in contact with the specimen. A method includes disposing a specimen within a recessed portion of a test apparatus and coupling a top plate of the test apparatus to a base of the test apparatus such that a sealing engagement is formed between the top plate and the specimen. The top plate defines a plurality of apertures, each of at least two of the apertures collectively with the specimen defines a well. A sample material can be disposed within at least one well.

### BACKGROUND

The disclosed invention relates generally to a test device and more particularly to a test device configured to provide high throughput cell-culture studies on biomaterials.

Known multi-well microplates, (e.g., 96-well plate, 384-well plate, 1536-well plate, and others in such a series of increasingly miniaturized well-sizes) are used for high-throughput studies for cell-culture and bioassays, as well as non-biological testing, such as residuals release and durability of materials. Such high-throughput testing approaches, however, have not been used for evaluation of biomaterials. Their application in cell-culture studies is more common for studying soluble molecules and drug-candidates (e.g., in the pharmaceutical industry). Typically, testing of biomaterials includes the use of individual sample coupons. For example, round 1 cm or 1.5 cm discs or coupons are constructed with a particular biomaterial and fitted within a multi-well microplate test assembly. Such test assemblies typically are each separately placed into wells of a cell-culture plate, presenting inherent disadvantages. For example, the individual coupons can be difficult to handle and have increased incidence of contamination and/or damage due to handling. Such damage and/or contamination can result in more variability and inaccuracies in test results. Moreover, artifacts can be introduced in the experimentation, such as cultured cells crawling under the samples from the edges, as well as other edge-effects (e.g., circular samples having different surface finishes and properties at their edge due to their processing).

Other problems associated with these small coupons are higher costs due to the inability to recycle damaged coupons, and an inefficient low-throughput process, as a coupon represents a single replicate of a biomaterial sample. In addition, the set-up time for testing with coupons is typically long, as each coupon is typically secured by a medical-grade silicon gasket and placed individually into a well on a microplate. The result can be long turn-around times per assay, and fewer replicates per assay. Moreover, high-throughput analytical methods cannot be effectively leveraged to complete efficient end-point analysis with the coupon or individual samples. Another consideration is that automation and robotic methods can be difficult to apply in this scenario. Because of these problems, and due to variation that can be generated between users, test standardization is also difficult. Typically, the statistical strength of the testing is also reduced since the number of replicates that can be accommodated is reduced.

Thus, a need exists for an apparatus and method for testing biomaterials that provides high-throughput capabilities, improved productivity, and improved accuracy of results.

### SUMMARY OF THE INVENTION

In one embodiment, a kit includes a base and a specimen removably couplable to the base. A top plate defines a plurality of apertures and is removably couplable to the base such that at least two of the apertures are associated with a specimen and each aperture defines a well with that specimen. Each well is configured to receive a sample material therein in contact with the specimen. A method includes disposing a specimen within a recessed portion of a test apparatus and coupling a top plate of the test apparatus to a base of the test apparatus such that a sealing engagement is formed between the top plate and the specimen. The top plate defines a plurality of apertures, each of at least two of the apertures collectively with the specimen defines a well. A sample material can be disposed within at least one well.

### BRIEF DESCRIPTION OF THE DRAWINGS

FIG. 1 is a schematic illustration of a test apparatus according to an embodiment of the invention.

FIG. 2 is a perspective view of a test apparatus according to an embodiment of the invention.

FIG. 3 is an exploded view of portions of the test apparatus of FIG. 2.

FIG. 4 is a top view of the test apparatus of FIG. 2.

FIG. 5 is a cross-sectional view of the test apparatus of FIG. 4 taken along line 5-5 in FIG. 4.

FIG. 6 is a detailed view of a portion A of the test apparatus of FIG. 5.

FIG. 7 is a perspective view of the test apparatus of FIG. 2 shown with a portion of the apparatus in cut-away.

FIG. 8 is a perspective view of a portion of the apparatus of FIG. 2.

FIG. 9 is a perspective view of a portion of the apparatus of FIG. 2.

FIG. 10 is a perspective view of a fixture according to an embodiment of the invention.

FIG. 11 is a perspective view of a reader tray according to an embodiment of the invention.

FIG. 12 is a perspective view of a test apparatus according to another embodiment of the invention.

FIG. 13 is a perspective view of a portion of the test apparatus of FIG. 12.

FIG. 14 is a perspective view of a test apparatus according to another embodiment of the invention.

FIG. 15 is a perspective view shown partially in cut-away of a portion of a test apparatus according to an embodiment of the invention.

FIG. 16. is a perspective view shown partially in cut-away of a portion of a test apparatus according to an embodiment of the invention.

FIG. 17 is a plan view of a specimen according to an embodiment of the invention.

FIG. 18 is a cross-sectional view of the specimen of FIG. 17, taken along line 18-18 in FIG. 17.

FIGS. 19-21 are plan views of various alternative embodiments of a specimen.

FIG. 22 is a flowchart illustrating a method according to an embodiment of the invention.

### DETAILED DESCRIPTION

The apparatuses and methods described herein can be used to test the effects of various sample materials on one or more specimens, such as a specimen constructed with a biomaterial. The apparatuses and methods provide for high-throughput testing and improved performance and accuracy over known test apparatuses and methods. For example, in some embodiments, the release of residuals, biological response and durability of variations on a single material or multiple materials can be tested within a single test and using multiple replicates. The specimen can be optionally coated with, for example, a polymer material, and the effects of various sample materials on the polymer material can be evaluated during a single test cycle. The apparatuses and methods described herein can be used for evaluation of both biomaterials (e.g., biocompatible materials) and material evaluation from non-biological perspectives. For example, the apparatuses and methods can be used to test metals, polymer-coated metals, compressed polymers and other materials used in medical device development. The apparatuses and methods can also be used to test the effects of sample materials on specimens constructed for example, with a mesh, fabric or film, or specimens including a liquid or therapeutic agent. A fixture is also described herein that can be used during a specimen coating procedure, such as spray-coating a polymer material onto a metal specimen.

As stated above, the apparatuses and methods described herein can be used for non-biological testing of materials (e.g. drug-release kinetics from polymers). Moreover, the invention can be applied to studying materials with a combinatorial approach (i.e. with a systematic variation in material samples along the testing-space on a high-throughput layout). Also, the apparatus is amenable to adapt to robotics, automation and high-throughput analytical methods.

In one embodiment, a kit includes a base and a specimen removably couplable to the base. A top plate defines multiple apertures and is removably couplable to the base such that at least two of the apertures are associated with a specimen, and each of the apertures can define a well with that specimen. Each well is configured to receive a sample material therein in contact with the specimen. A method according to an embodiment of the invention includes disposing a specimen within a recessed portion of a test apparatus and coupling a top plate of the test apparatus to a base of the test apparatus such that a sealing engagement is formed between the top plate and the specimen. The top plate defines a plurality of apertures; each of at least two of the apertures collectively with the specimen defines a well. A test material can be disposed within at least one well.

The term “specimen” is used herein to mean an item having a surface formed of a material to be evaluated. A specimen can take one of a variety of different shapes, sizes and forms. A specimen can also have a variety of different types of surfaces to be tested. For example a specimen can have a planar surface, a curved surface, a textured or roughened surface, etc. A specimen can be a variety of different shapes, such as, for example, oval, round, rectangular, elliptical, semi-circular, or diamond shaped. For example, a specimen can be an elongated strip of material. A specimen can also be formed with one or more of a variety of different materials as will be described in more detail below.

The term “sample material” (also referred to herein as “test material”) is used herein to mean a substance, material, component, dye, cell, biological material, non-biological material, plant matter, etc. that can be disposed within a well of a test apparatus adjacent to and/or in contact with a surface of a specimen. In some embodiments, the sample material is a biological material and the effects of the sample material on the specimen can be tested. In other embodiments, the sample material is a non-biological material, such as a buffer solution, that is used to evaluate the rate of elution of an agent, such as a pharmaceutical, from a polymer that is coated on a specimen.

The term “biomaterial” is used herein to mean a biocompatible material used for the construction of medical devices or material being developed for use in the construction of medical devices.

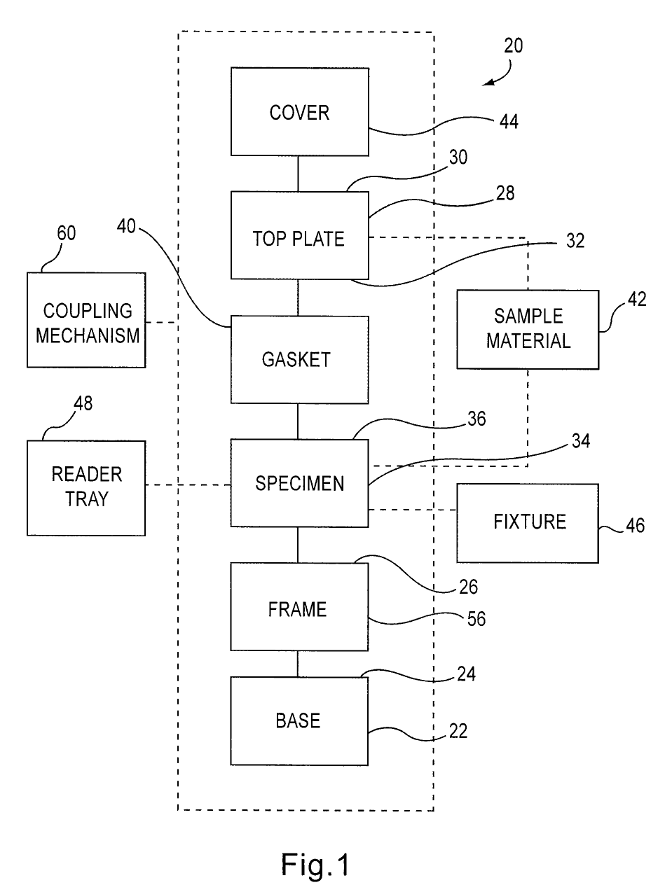

FIG. 1 is a schematic illustration of an apparatus according to an embodiment of the invention. A test apparatus 20 includes a base 22 and a top plate 28. The base 22 has an upper face 24, and the top plate 28 has an upper face 30 and a lower face 32. The top plate 28 can be removably coupled to the base 22 with a coupling mechanism 60. The top plate 28 defines multiple apertures (not shown in FIG. 1) that extend between the upper face 30 and the lower face 32 of the top plate 28. The apertures can be a variety of different shapes and sizes. For example, the apertures can be round, oval, square, or rectangular. In some embodiments, the apertures are configured as elongated slots.

The test apparatus 20 also includes one or more specimens 34 that can be removably received within a recessed portion defined by a frame 56 that is disposed between the base 22 and the top plate 28. The recessed portion of the frame 56 includes a surface countersunk from an upper face 26 of the frame 56. The specimens 34 can be disposed on the frame 56 such that an upper surface 36 of the specimen 34 is substantially flush with, lower than, or extending above the upper face 26 of the frame 56. In some embodiments, the test apparatus 20 does not include a separate frame 56. In such an embodiment, the base 22 can define a recessed portion having a surface countersunk from the upper face 24 of the base 22 configured to receive one or more specimens 34.

When the top plate 28 is coupled to the base 22, an upper surface 36 of the specimen 34 is associated with one or more apertures of the top plate 28 and defines a well with each aperture (not shown in FIG. 1). Thus, each specimen 34 is associated with multiple wells. The wells are open at the upper face 30 of the top plate 34 to allow for the insertion of a sample material within the wells, as will be described in more detail below. The depth or thickness of the top plate 28 can be varied to provide for deeper or shallower wells.

The lower face 32 of the top plate 28 can optionally be formed with a material to provide a sealing fit between the top plate 28 and the specimen 22. Thus, each of the wells defined by the apertures and the specimen 34 will be sealed at the lower face 32 of the top plate 28. In other embodiments, the test apparatus 20 can include a gasket 40 to provide a sealing fit between the top plate 28 (and the apertures on the top plate 28) and the upper surface 36 of the specimens 34. The gasket 40 can be removably disposed between the frame 56 and the top plate 28 (e.g., on the upper face 26 of the frame 56). The gasket 40 defines openings (not shown in FIG. 1) that extend from a top face of the gasket 40 to a bottom face of the gasket 40. The openings of the gasket 40 can correspond to, and align with, the apertures of the top plate 28.

The specimens 34 can be formed with various materials and compositions, such as, for example, biomaterials such as metals (e.g., stainless steel, titanium), polymers (e.g., compressed polymers), polymer-coated metals, polymer-coated plastics, and other materials used in the development of medical devices. The specimens 34 can be substantially planar and rigid. In alternative embodiments, the specimens 34 can be flexible and include, for example, films of various compositions, such as films that are sprayed, molded or cast. The specimens 34 can also be formed with meshes or fabrics, glass, plant matter or minerals, or with various biological materials such as, for example, bone, tissue, cartilage, and cell sheets. The specimens 34 can also include a material such as a liquid film, or a material saturated or including a therapeutic agent. The specimens 34 can have a smooth or a polished surface to be tested, or alternatively have a treated surface, such as a surface that has been, for example, sanded, plasma treated, or bead blasted.

When assembled, the test apparatus 20 can be used to evaluate the effects of various sample or test materials on one or more specimens 34. For example, one or more sample materials 42 can be disposed within one or more of the wells such that the sample materials 42 are adjacent to or in contact with a specimen 34 associated with the well(s). The sample material(s) 42 can include, for example, fluids, cells, plant matter, etc. Other materials can be disposed within a well such as, for example, a dye that can be used to test the integrity of the seal between the specimen 34 and the top plate 28. After the sample material 42 is disposed within the wells, a cover 44 can optionally be placed over the top plate 28 to seal the wells at the top face 30 of the top plate 28. The wells can be covered or sealed collectively, or individually. In some embodiments, a cover 44 can be in the form of a film. In other embodiments, the cover 44 can be a rigid or flexible lid.

After the sample material(s) 42 has been disposed within the wells, the sample material(s) 42 can then be incubated for a selected period of time (“test cycle”) and the effects of the sample material(s) 42 on the specimen 34 can then be evaluated after the designated test cycle has ended. The test apparatus 20 can be used to test the effects of biological materials (e.g., cell-culture, cell-growth, cell-interaction, bioassays) and non-biological materials on both biomaterials and non-biomaterials.

In some embodiments, the test apparatus 20 can be used to test the kinetics of drug release (“KDR”). For example, the rate of release of a drug (e.g., a therapeutic agent) disposed, embedded, saturated, etc. within a coating (e.g., a polymer coated on a surface of a specimen) on a specimen, or from a polymer specimen, can be tested and evaluated. For example, a stainless steel specimen can have a polymer coating disposed on its surface and a therapeutic agent can be disposed within the polymer. A sample material in the form of, for example, a liquid, or other buffer solution, can be disposed in one or more wells. The buffer can be, for example, a phosphate buffered saline (“pbs”). As the polymer breaks-down (e.g., deteriorates, degrades, etc.) and the therapeutic agent is eluted from the polymer, the eluted portions will be disposed within the buffer solution. The buffer solution can be tested and analyzed to determine the rate of release of the therapeutic agent from the polymer. The test apparatus 20 can also be heated, cooled, or agitated to induce drug release from the specimen. Samples can be taken from the well, or sensors can be used to analyze the well contents and monitor the drug release from the specimen. The rate of degradation of the coating or material on the specimen (e.g., the polymer) can also be tested.

Thus, a test apparatus according to the invention can be used for a variety of different test applications. For example, a test apparatus can be used for testing the effects of biological and non-biological materials on a specimen, testing drug-elution from a polymer specimen or a polymer coated specimen, or testing the specimen itself, for example, the mechanical properties of a polymer specimen, testing for leachables, biocompatibility of a specimen, etc. The test apparatus and methods described herein provide a high-throughput and efficient test process, which can also be used to leverage subsequent testing.

The test apparatus 20 can be used to test multiple different specimens 34 within a single test cycle. In addition, because a single specimen 34 is associated with multiple wells, multiple different sample materials can be tested on a single specimen 34. The ability to test and compare the effects of multiple sample materials on a single specimen 34 reduces the variability and inaccuracies that often occur when each sample material is tested on a different single test coupon. In addition, the specimens 34 are larger in size than a typical test coupon, and therefore, easier to handle during insertion and removal of the specimens 34 to and from the test apparatus 20. This easier handling enables a user to remove the specimens 34 after the test cycle so that the specimens 34 can be further evaluated, for example, on a device such as a spectrophotometer.

As described above, some specimens may be coated with for example a polymer. The configuration of the specimens 34 allow them to be easily coated with a polymer, or other material, prior to testing the specimen 34 in test apparatus 20. In some embodiments, a fixture 46 can be used to hold the specimens 34 during a coating process. The use of a fixture 46 further improves the process of coating the specimens 34, and reduces damage and contamination that can occur when coating smaller coupons.

In some embodiments, the test apparatus 20 can be included in a kit that includes a reader tray 46. The reader tray 46 can define a recessed portion similar to the frame 56 such that the specimens 34 can be placed within the reader tray 46 after the test cycle. In some embodiments, the reader tray can be configured to receive the frame 56 therein, with the specimens disposed within the recessed portion of the frame 56. The reader tray 46 provides a clean and efficient method for transporting the specimens 34 from, for example, the test apparatus 20 to a spectrophotometer or other device for further evaluation.

The top plate 28, the base 22, the frame 56, and the gasket 40 can each be formed with various materials, preferably materials that can be sterilized for repeated use, such as stainless steel and silicone rubber. The top plate 28 can be configured to meet the ANSI standard for well plates (e.g., ANSI/SBS 1-2004, 2-2004, 3-2004, 4-2004), which will allow the test apparatus 20 to be used with existing automated testing equipment. The various components of the test apparatus 20 are removably coupled together, allowing for the test apparatus 20 to be disassembled for washing and sterilizing, and reassembled as desired.

Having described above various general principles, several example embodiments are now described. These embodiments are only examples, and many other configurations of test apparatus 20 and its various components are contemplated, and will be apparent to the artisan in view of the general principles described above and the exemplary embodiments.

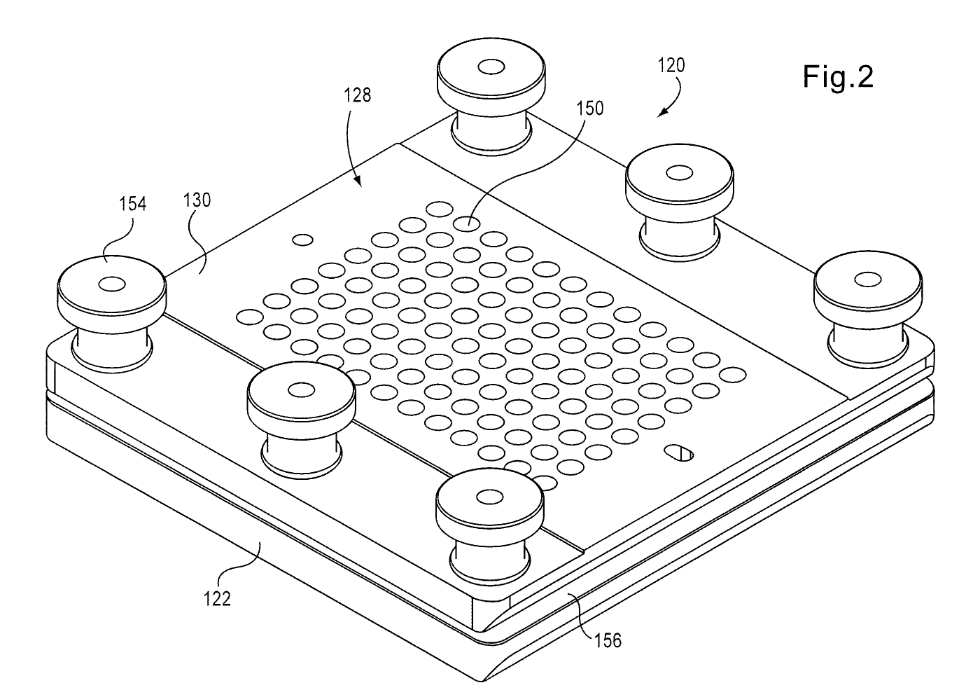

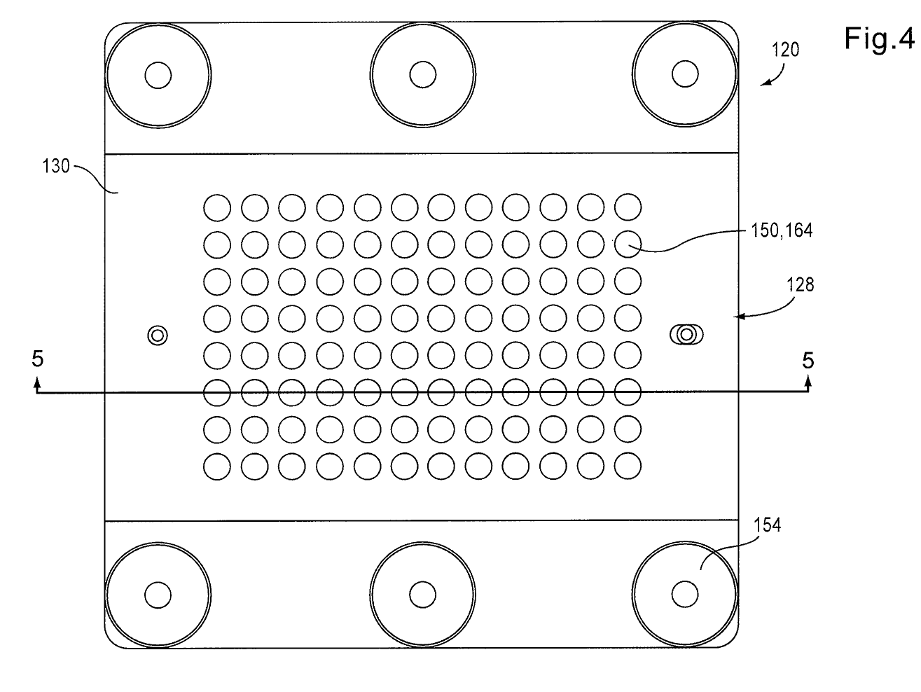

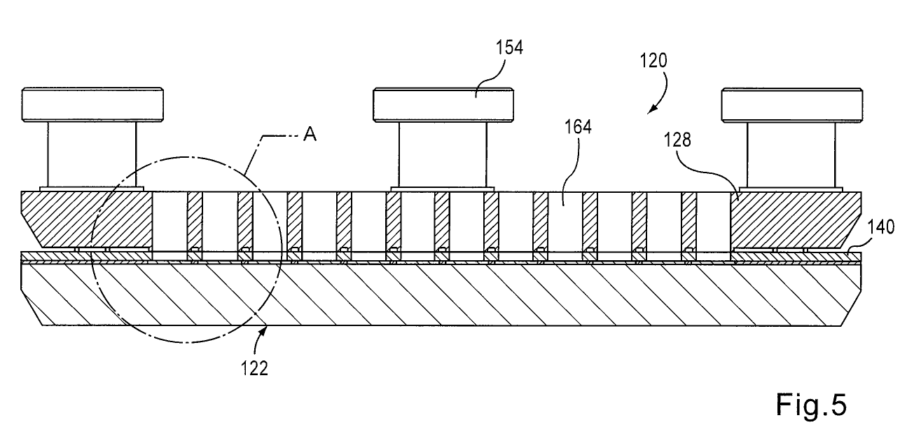

FIGS. 2-9 illustrate a test apparatus according to an embodiment of the invention. A test apparatus 120 includes a base 122, a frame 156, a top plate 128, a gasket 140 and multiple specimens 134, each in the shape and form of an elongated strip. The top plate 128 includes an upper face 130, a lower face 132, and defines multiple apertures 150 extending from the upper face 130 to the lower face 132.

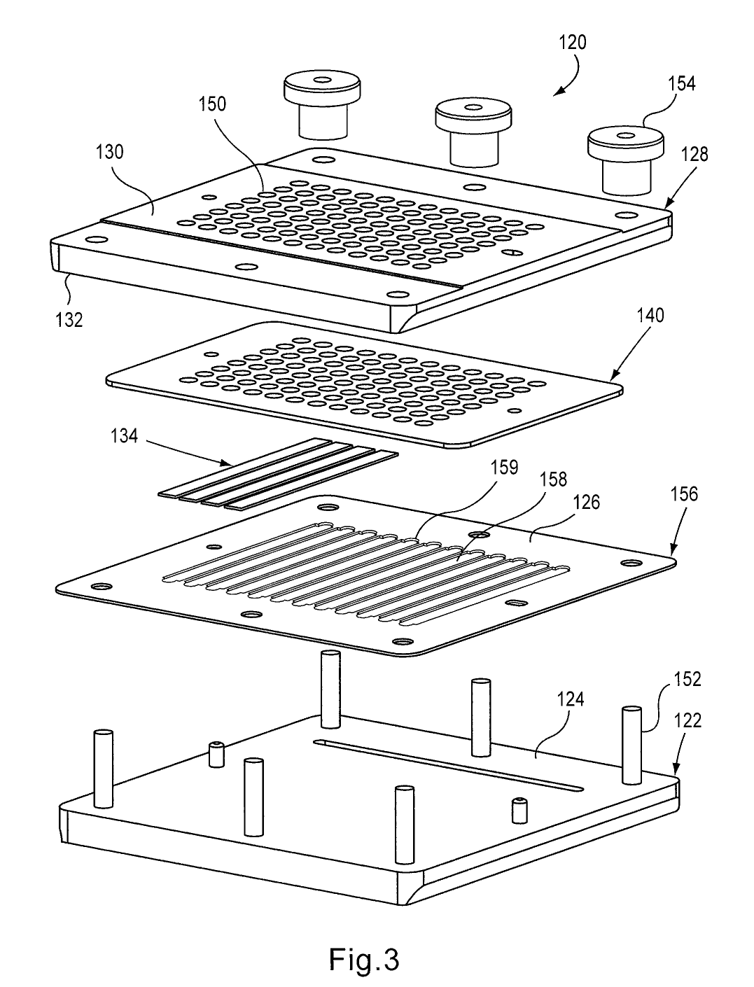

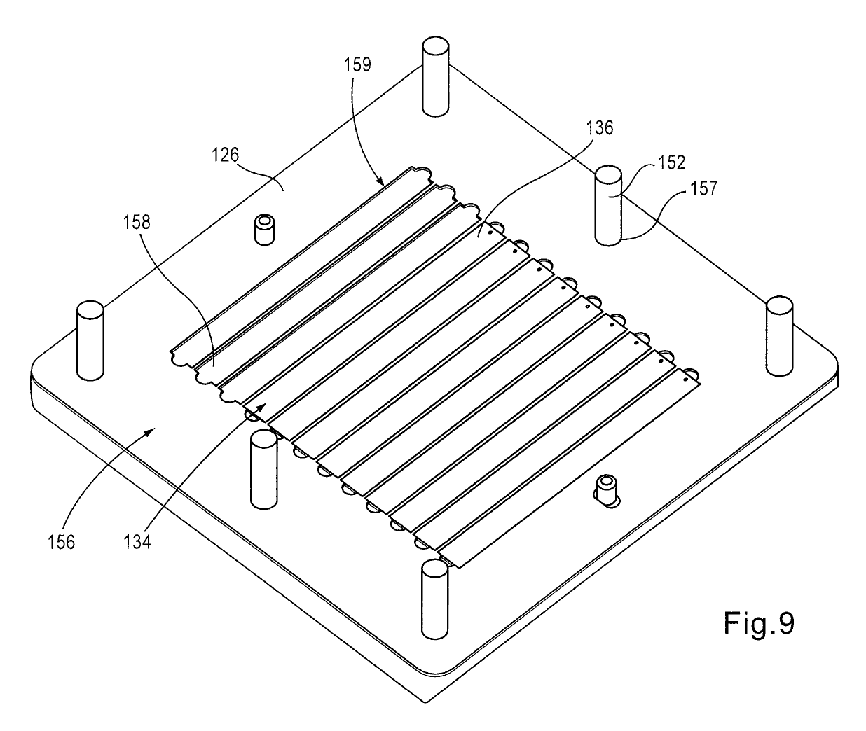

The base 122 includes an upper face 124 (FIG. 3) to which the frame 156 is coupled. The frame 156 defines a recessed portion 159 having a counter surface 158 that is configured to receive multiple specimens 134. FIG. 3 is an exploded view of portions of the test apparatus 120 and FIG. 9 illustrates a portion of the test apparatus 120 without the top plate 128 showing a portion of the frame 156 with specimens 134 disposed within the recessed portion 159, and a portion of the frame 156 without specimens 134. The countersunk surface 158 allows specimens 134 to be disposed within the recessed portion 159 of the frame 156 such that an upper surface 136 of the specimens 134 is substantially flush with the upper face 126 of the frame 156.

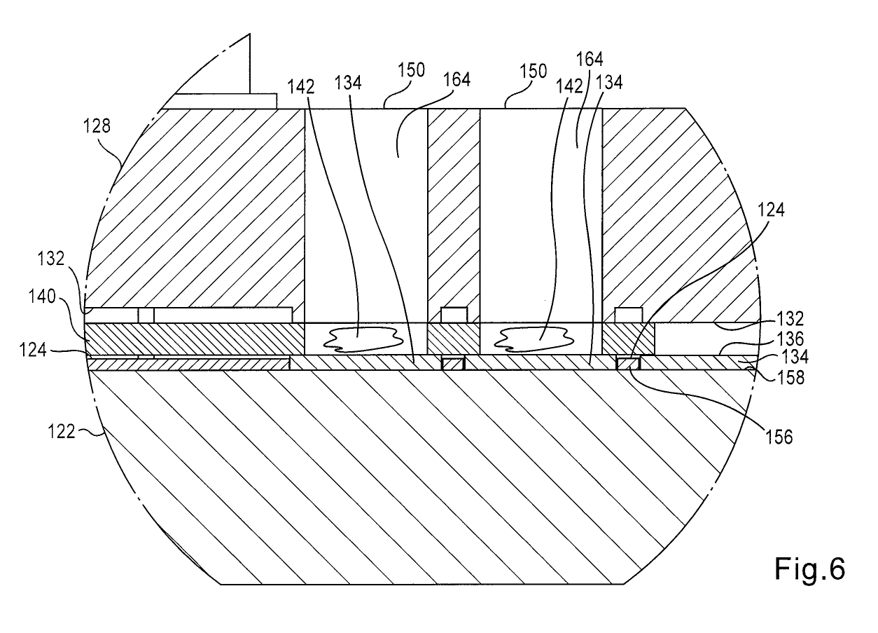

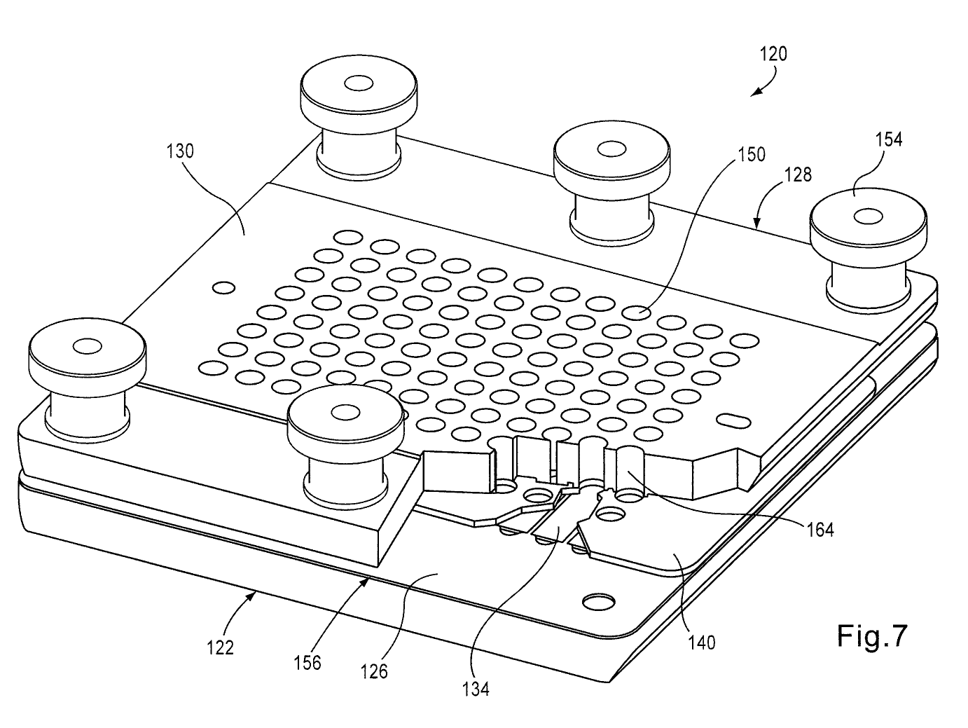

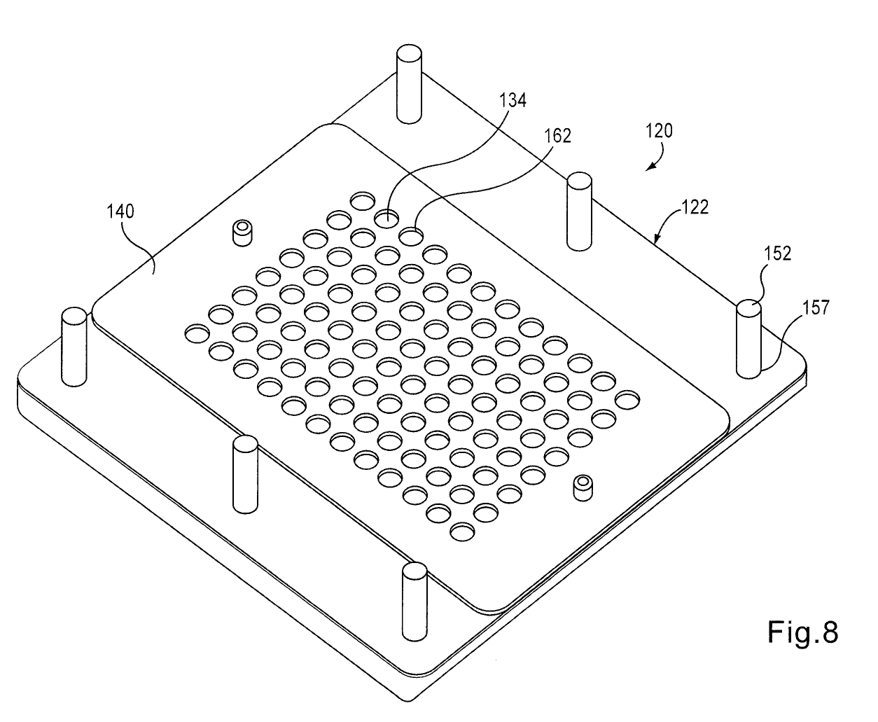

A gasket 140 can be disposed between the frame 156 and the top plate 128, as shown in FIG. 8. FIG. 8 illustrates a portion of the test apparatus 120 with the gasket 140 disposed on the frame 156 over the specimens 134. The gasket 140 defines multiple openings 162 that align with, and correspond to, apertures 150 of the top plate 128 when the top plate 128 is coupled to the base 122, as shown in the partial cut-away view of FIG. 7 and in the detailed view of FIG. 6. When the top plate 128 is coupled to the base 122, multiple wells 164 are defined by the apertures 150 of the top plate 128 and each of the specimens 134. Thus, each specimen 134 is associated with multiple different apertures 150. The gasket 140 provides a sealing fit between the top plate 128 and the specimens 134, providing a seal at a bottom portion of the wells 164.

In this embodiment, the coupling mechanism by which the top plate 128 is removably coupled to the base 122 includes a set of pins 152 (FIGS. 3, 8 and 9) that extend upward from the base 122 and through openings 157 defined by the frame 156, and that are received in mating openings (not shown) defined by the top plate 128. A nut 154 can be threaded on to each pin 152 to couple the top plate 128 to the base 122, sandwiching the gasket 140 and the specimens 134 there between.

As shown in FIG. 6, after the top plate 128 is coupled to the base 122, one or more test or sample materials 142 can be disposed within one or more of the wells 164 to test the effects of the sample material(s) 142 on the specimen(s) 134. For example, the sample material(s) 142 can be incubated for a selected period of time and the specimens 134 can then be evaluated to determine the effects of the sample material(s) 142 on the specimens 134. As shown in FIG. 7, there are multiple wells 164 associated with a single specimen 134. This configuration allows for multiple tests to be performed on a single specimen 134 during a single test cycle. For example, a different test material 142 can be disposed within each of the different wells 164 associated with a particular specimen 134.

As stated above, in some embodiments it may be desirable to apply a coating or layer of material to a surface of a specimen prior to placing the specimen within the base 122. FIG. 10 illustrates an embodiment of a fixture 146 that can be used during a procedure to apply such a layer or coating of material to a specimen. The fixture 146 can include a cylindrical frame 166 similar to the frame 156, and the specimens 134 can be received within a recessed portion of the cylindrical frame 166. A pair of clamps 168 can be used to secure the specimens 134 within the cylindrical frame 166. A coating or layer of material can then be applied on at least a portion of the surface 136 of the specimens 134.

Also as stated above, in some embodiments, a reader tray can be provided. A reader tray 148 can define a frame 170 similar to the frame 156 configured to receive the specimens 134 therein, as shown in FIG. 11. After the sample material(s) 142 has incubated for the selected test cycle time within the test apparatus 120, the specimens 134 can be removed from the test apparatus 120 and placed within the reader tray 148. The reader tray 148 can be used to transport the specimens 134 to other locations to be evaluated with other devices such as, for example, a spectrophotometer. The reader tray 148 can also be used to support the specimens 134 during evaluation.

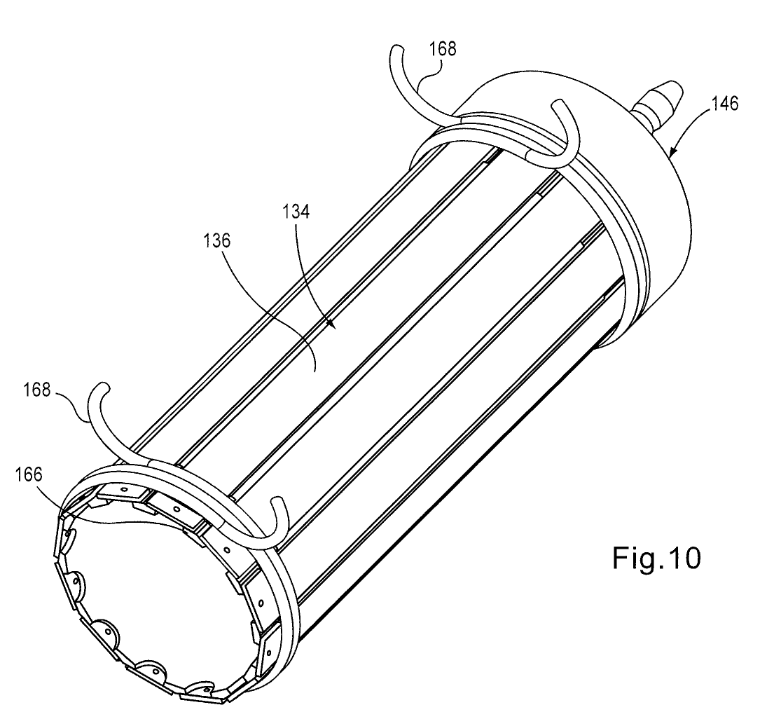

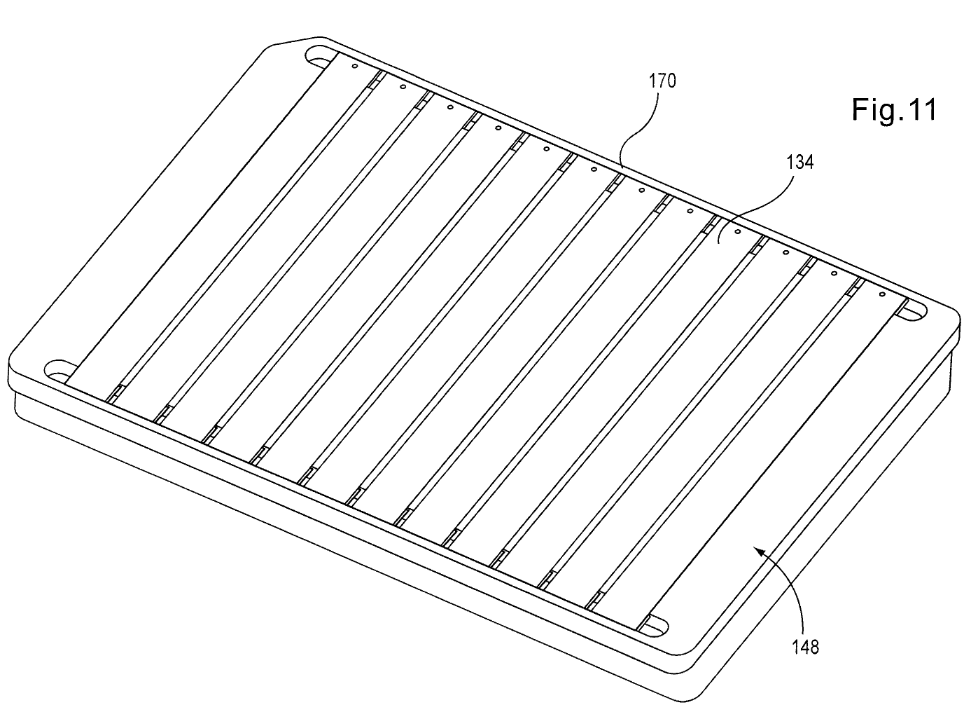

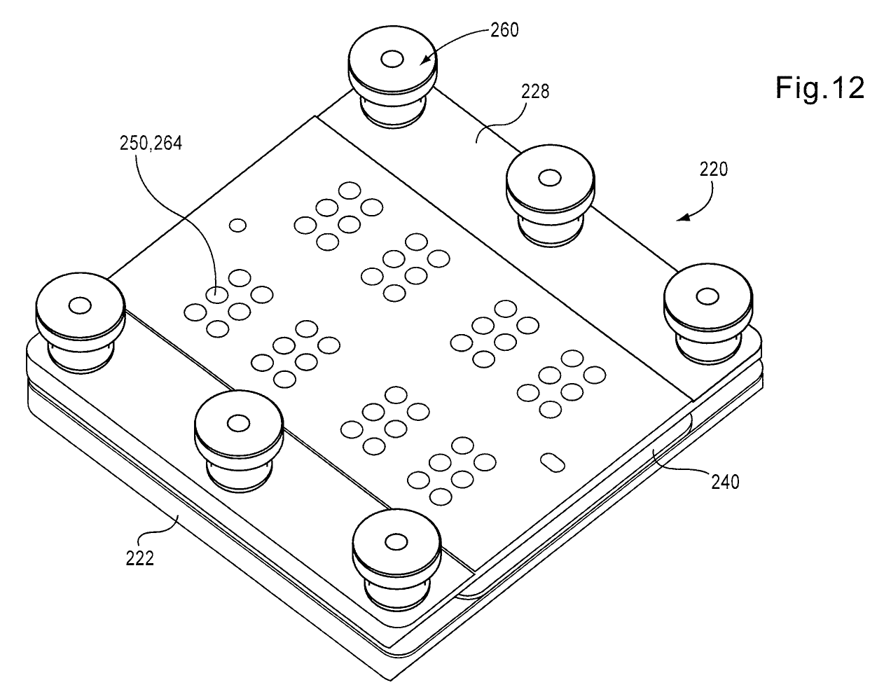

FIGS. 12 and 13 illustrate a test apparatus according to another embodiment of the invention illustrating a different pattern of apertures defined by a top plate and a different shape and size of specimens. A test apparatus 220 includes a base 222, a top plate 228 and multiple specimens 234. FIG. 12 is a perspective view of the test apparatus 220 with the top plate 228 coupled to the base 222, and FIG. 13 is a perspective view of the test apparatus 220 with the top plate 228 removed. The test apparatus 220 is similar to the previous embodiment and can perform the same functions.

In this embodiment, the base 222 defines a recessed portion 261 having a countersunk surface 263 and that is configured to receive specimens 234, as shown in FIG. 13. FIG. 13 shows one specimen 234 removed from the recessed portion 261 for illustration purposes. The top plate 228 defines multiple apertures 250 and can be coupled to the base 222 with a coupling mechanism 260, as described in the previous embodiment. When the top plate 228 is coupled to the base 222, the apertures 250, collectively with the specimens 234, define multiple wells 264. In this embodiment, the apertures 250 are arranged in groups or clusters of 6 apertures 250, rather than in rows as illustrated in the previous embodiment. Each of the specimens 234 is configured to correspond with one of the clusters of apertures 250. As with the previous embodiments, one or more sample materials (not shown) can be disposed within the wells 264 and incubated for a selected period of time. Thus, multiple different sample materials 242 can be tested on a single specimen 234.

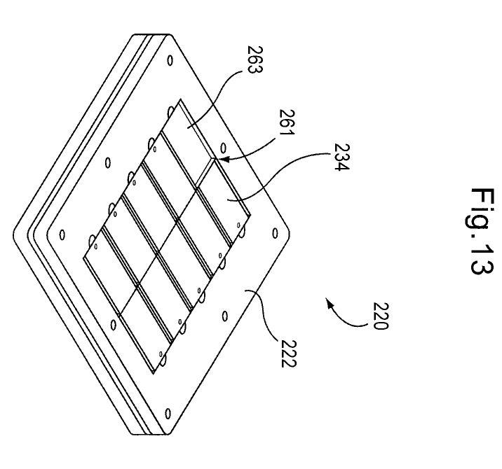

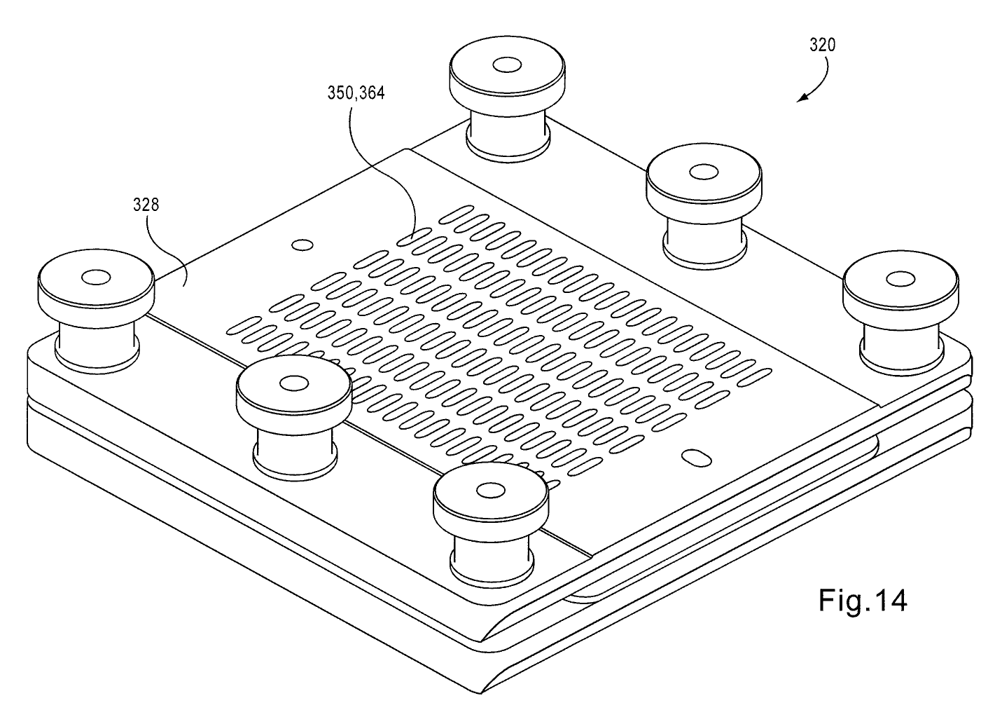

FIG. 14 is a perspective view of a test apparatus 320 according to another embodiment of the invention. The test apparatus 350 is similar to the previously described embodiments except the test apparatus 350 includes a top plate 328 that defines apertures 350 and wells 364 that are oblong or oval shaped. The test apparatus 320 can be formed in the same manner and be used to perform the same functions as described above for the previous embodiments.

In some embodiments, a test apparatus according to the invention can be used to test layered materials. For example, a specimen can be coated with a layer of material, and then a shim or gasket, a mesh, fabric and/or a second coated specimen. These layers of various specimens, films, meshes, plant/animal matter, fabrics, spaces and liquids can be used to simulate material and drug interactions with cells in a lab environment.

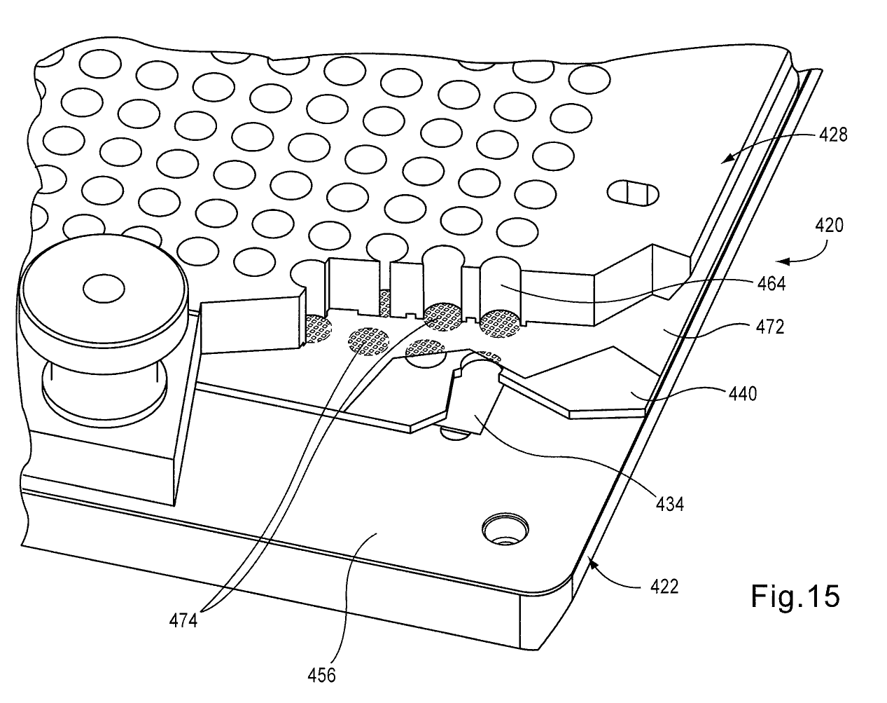

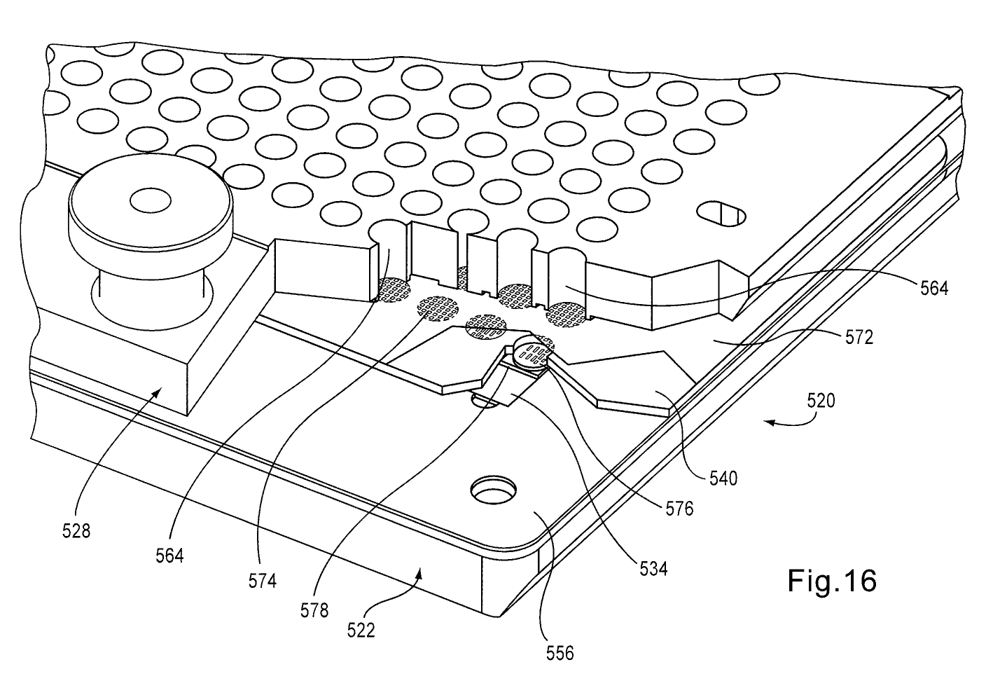

FIGS. 15 and 16 are each perspective views of a portion of an embodiment of a test apparatus that each illustrate a layered test configuration. As shown in FIG. 15, a test apparatus 420 includes a base 422, a frame 456, a gasket 440 and a top plate 428. In this embodiment, the test apparatus 420 includes a mesh plate 472 (shown partially cut-away) disposed between the top plate 428 (shown partially cut-cutaway) and the gasket 440 (shown partially cut-away). The mesh plate 472 defines multiples mesh portions 474 that are positioned to correspond to multiple wells 464 defined by the top plate 428. As with the previous embodiments, the test apparatus 420 is configured to receive multiple substrates 434 within a recessed portion of the frame 456 (not shown in FIG. 15). The mesh plate 472 is one example of an additional layer of material that can be tested. For example, a test sample of material (not shown) can be placed within a well 464 and the effects and/or interaction between the test sample, the mesh plate 472 and a substrate 434 can be tested and evaluated.

A test apparatus 520, illustrated in FIG. 16 includes a base 522, a frame 556, a gasket 540 and a top plate 528 that defines multiple wells 564. A mesh plate 572 (shown partially cut-away) is disposed between the gasket 540 (shown partially cut-away) and the top plate 528 (shown partially cut-away) and one or more first substrates 534 are disposed within a recessed portion of the frame 556. In this embodiment, one or more second substrates 576, having a substantially planar configuration and defining apertures therethrough, are disposed beneath the gasket 540. As shown in FIG. 16, the second substrates are circular shaped and correspond to the shape of the multiple wells 564, however, the second substrates can have other configurations not specifically shown. For example, the second substrates 576 can be elongated and correspond to multiple different wells 564. In addition, a test material 578, such as animal tissue is disposed between the second substrate 576 and the first substrate 534. This embodiment is another illustration of how multiple different substrates, test materials, etc. can be coupled in a layered configuration to test the effects of each has on the other, and/or to test the effects of another sample material (not shown) disposed within the wells 564 on the various layered components.

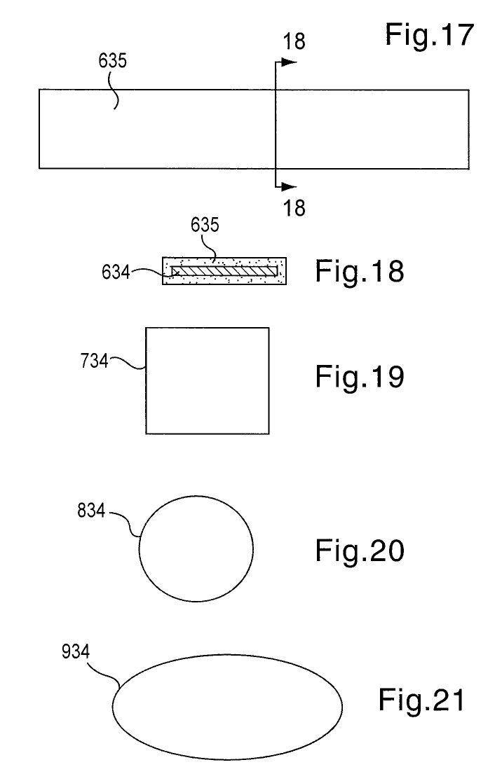

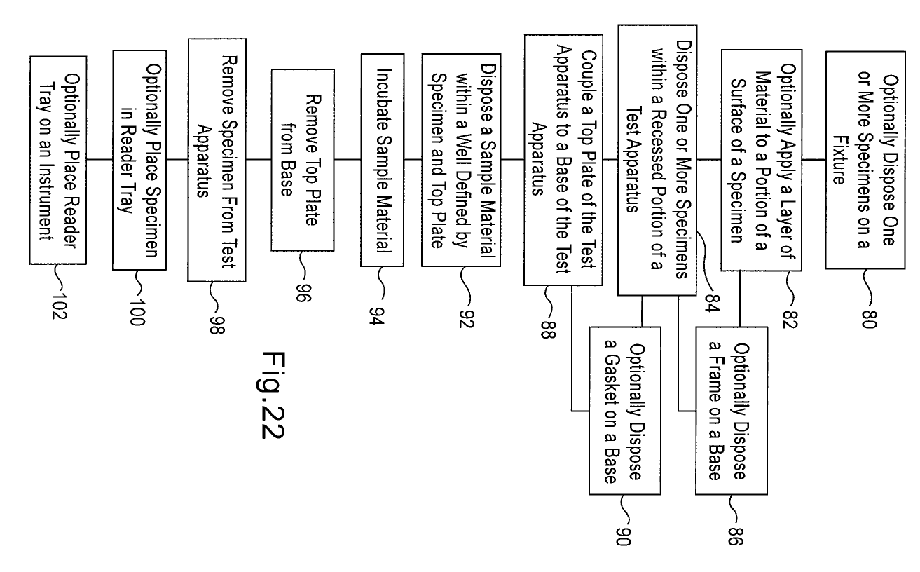

As stated above, the specimens can be a variety of different shapes and sizes. FIGS. 17-21 illustrate various different example embodiments of a specimen. FIGS. 17 and 18 illustrate a substantially elongated and substantially rectangular specimen 634, having a coating 635 disposed thereon. For example, the coating 635 can be a polymer having an agent, such as a pharmaceutical drug, disposed within the polymer to test, for example, the drug-elution rate from the polymer as described above. FIG. 19 illustrates a specimen 734 that is substantially square. Although specimens 634 and 734 are shown with sharp corners, the specimens 634 and 734 can alternatively have rounded corners. FIG. 20 illustrates a specimen 834 that is substantially circular, and FIG. 21 illustrates a specimen 934 that is substantially oval. In any embodiment, the specimen can have a variety of different lengths, widths, and thicknesses.

FIG. 22 is a flow chart of a method according to an embodiment of the invention. A method includes optionally coupling one or more specimens within a test fixture as shown at 80. In some embodiments, at least a portion of the specimen is formed with a biomaterial. At 82, a layer of material can optionally be applied to at least a portion of a surface of at least one of the one or more specimens. Such a layer of material can be applied to the surface of a specimen while the specimen is disposed within a test fixture, or alternatively when the specimen is not disposed within a test fixture. One or more specimens are disposed within a recessed portion of a base or a frame coupled to the base, at 84. For example, a frame can optionally be disposed on an upper face of the base prior to disposing a specimen, at 86. A top plate is coupled to the base at 88 such that a sealing engagement is formed between the top plate and the specimen.

In some embodiments, prior to coupling the top plate to the base, a gasket can be disposed between the base and the top plate, at 90. The gasket can provide a sealing fit between the top plate and the specimen. The top plate defines a plurality of apertures. Each aperture from the plurality of apertures collectively with a specimen defines a well. A sample or test material is disposed within at least one of the wells at 92. The sample material can be, for example, a biological material, a non-biological material, a fluid, a dye, a cell, etc. At 94, the sample material is incubated for a predetermined time period. At 96 the top plate can be removed from the base, and at 98 the specimen can be removed from the test apparatus. At 100, the specimen can optionally be placed in a reader tray after being removed from the test apparatus. The reader tray can then be placed on or within another instrument, such as a photospectrograph, at 102.

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

The previous description of the embodiments is provided to enable any person skilled in the art to make or use the invention. While the invention has been particularly shown and described with reference to embodiments thereof, it will be understood by those skilled in art that various changes in form and details may be made therein without departing from the spirit and scope of the invention.

For example, the various features of a test apparatus 20 (120, 220, 320, 420, 520) may include other configurations, shapes and materials not specifically illustrated, while still remaining within the scope of the invention. Thus, a test apparatus can include various combinations and/or sub-combinations of the various features and/or components described in the various embodiments herein. Also, any number of different layers of substrates, sample materials, plates, tissue, meshes, films, coatings, etc. can be tested with a test apparatus described herein.

What is claimed is:

1. A kit, comprising:
a base;
a specimen removably couplable to the base; and
a top plate defining a plurality of apertures and removably couplable to the base such that at least two apertures from the plurality of apertures are associated with a specimen and define a well with that specimen, each well being configured to receive a sample material therein in contact with the specimen.

2. The kit of claim 1, wherein with the sample material disposed within a well, the effects of the sample material on the specimen over a selected period of time can be tested.

3. The kit of claim 1, wherein the sample material is a buffer material and a coating is disposed on a surface of the specimen, the coating having an agent embedded therein such that the rate of release of the agent from the coating can be tested when the specimen is coupled to the base and the sample material is disposed within the wells.

4. The kit of claim 1, further comprising:
a frame couplable to an upper face of the base and defining a recessed portion, the specimen being removably receivable within the recessed portion of the frame.

5. The kit of claim 1, wherein the base defines a recessed portion, the specimen being removably receivable within the recessed portion of the base.

6. The kit of claim 1, wherein at least a portion of the specimen is formed with a biomaterial.

7. The kit of claim 1, wherein the sample material receivable in at least one of the wells is a biological material.

8. The kit of claim 1, wherein the specimen is an elongated strip.

9. The kit of claim 1, further including a gasket removably disposable between the base and the top plate to form a sealing engagement between the top plate and the specimen.

10. The kit of claim 1, wherein the specimen is a first specimen from a plurality of specimens removably disposable on the base, at least two additional apertures from the plurality of apertures is associated with each specimen from the plurality of specimens and defines a well with that specimen.

11. The kit of claim 1, wherein the top plate is a first top plate from a plurality of top plates removably couplable to the base and the specimen is a first specimen from a plurality of specimens removably disposable on the base.

12. The kit of claim 1, wherein the specimen includes a first portion formed of a first material and a second portion formed of a second material.

13. An apparatus, comprising:
a base;
a frame; and
a top plate removably couplable to the base, the top plate defining a plurality of apertures, the frame configured to receive a specimen that defines multiple wells with at least two of the plurality of apertures when the top plate is coupled to the base, the wells being configured to receive a sample material therein in contact with the specimen to test the effects of the sample material on the specimen.

14. The apparatus of claim 13, further comprising:
a gasket removably couplable to the frame between the base and the top plate to form a sealing engagement between the top plate and a specimen.

15. The apparatus of claim 13, wherein the frame is configured to receive a specimen formed at least in part with a biomaterial.

16. The apparatus of claim 13, wherein the frame is configured to receive a plurality of specimens.

17. The apparatus of claim 13, wherein the frame is configured to receive a specimen having a first material portion and a second material portion.

18. The apparatus of claim 13, wherein a sample material disposable in a well includes a biological material.

19. The apparatus of claim 13, wherein each well from the at least two wells is configured to receive a different sample material therein to test the effects of the sample material on an associated specimen.

20. The apparatus of claim 13, wherein the frame is configured to receive a plurality of specimens, at least one specimen from the plurality of specimens is formed with a different material than another specimen from the plurality of specimens.

21. The apparatus of claim 13, wherein a specimen receivable on the frame is an elongated strip.

22. A method, comprising:
disposing a specimen within a recessed portion of a test apparatus;
coupling a top plate of the test apparatus to a base of the test apparatus such that a sealing engagement is formed between the top plate and the specimen, the top plate defining a plurality of apertures, each of at least two apertures from the plurality of apertures collectively with the specimen defines a well; and
disposing within at least one well a sample material.

23. The method of claim 22, further comprising:
incubating the sample material for a predetermined time period.

24. The method of claim 22, further comprising:
removing the top plate from the base; and
removing the specimen from the base.

25. The method of claim 22, further comprising:
prior to disposing the specimen, disposing the specimen on a fixture.

26. The method of claim 22, further comprising:
prior to disposing the specimen, applying on at least a portion of a surface of the specimen a selected layer of material.

27. The method of claim 22, wherein the sample material includes a biological material.

28. The method of claim 22, wherein at least a portion of the specimen is formed with a biomaterial.

29. The method of claim 22, wherein the disposing a specimen includes disposing a plurality of specimens within the recessed portion of the test apparatus, each specimen from the plurality of specimens being associated with at least two apertures from the plurality of apertures.

30. The method of claim 22, further comprising:
prior to the coupling the top plate to the base, disposing a gasket between the base and the top plate providing a sealing fit between the top plate and the specimen.

31. The method of claim 22, further comprising:
prior to disposing the specimen, disposing a frame defining the recessed portion on an upper face of the base.

32. The method of claim 22, wherein the disposing the specimen includes disposing the specimen within a recessed portion defined by the base.
